(19) United States
(12) Patent Application Publication
(10) Pub. No.: US 2009/0042770 A1
(43) Pub. Date: Feb. 12, 2009

(54) Branched Chain Amino Acid Chelate

(76) Inventors: Eric D. Bastian, Twin Falls, ID (US); Brent P. Hagen, Birgham City, UT (US)

Correspondence Address: DONNA J. RUSSELL, 1492 ANTHONY WAY, MT. JULIET, TN, 37122, US

(21) Appl. No.: 12/188,172

(22) Filed: Aug. 7, 2008

Related U.S. Application Data
(63) Continuation-in-part of application No. 12/014,065, filed on Jan. 14, 2008.
(60) Provisional application No. 60/884,890, filed on Jan. 14, 2007.

(51) Int. Cl. A61K 31/195; A61K 38/00

(52) U.S. Cl. 514/2; 514/561

(57) ABSTRACT

Compositions are disclosed comprising mineral/branched-chain amino acid chelates such as, for example, calcium leucinate, for increasing absorption of the branched-amino acid in a human or animal subject. Methods are also provided for using the compositions to increase branched-chain amino acid absorption to increase or maintain muscle mass in athletes or in individuals having diseases such as ALS, muscular dystrophy, sarcopenia associated with aging, or muscle atrophy associated with spinal cord injury, as well as to decrease mental fatigue.

This is a continuation-in-part of U.S. patent application Ser. No. 12/014,065, which claims the benefit of priority of U.S. provisional patent application No. 60/884,890, filed Jan. 14, 2007.

### FIELD OF THE INVENTION

The present invention relates to nutritional compositions. More specifically, the invention relates to dietary supplements for increasing branched-chain amino acid delivery and for methods of providing increased levels of branch-chain amino acids utilizing dietary supplements.

### BACKGROUND OF THE INVENTION

Mineral absorption generally occurs in the small intestine via a combination of active, or transcellular, absorption and passive, or paracellular, absorption. A variety of factors, including undesirable chelation reactions (e.g., reaction with another mineral) in the digestive system, may make dietary minerals more difficult for the body to absorb. To address this problem, nutritional supplementation of foods with minerals has become common and a primary method for providing minerals in foods and in supplements is to provide them as chelates formed with amino acids or peptides to improve absorption of the mineral.

Minerals provided in the chelated form are more available for absorption. Studies in animals, for example, have demonstrated that chelated minerals provided in the diet result in higher mineral absorption and utilization.

The American Association of Feed Control Officials (AAFCO) defines a chelate as a product resulting from the reaction of a metal ion from a soluble metal salt with amino acids having a mole ratio of one mole of metal to one to three (preferably 2) moles of amino acids to form coordinate covalent bonds. Metal/amino acid chelates for improving absorption of minerals have been described previously, where the metal is chelated with an alpha amino acid (e.g., glycine, lysine, etc.). For example, U.S. Patent application publication number 2002/0058088 (Henry et al.) describes chelates of alpha-amino acids for producing color stable iron-fortified compositions, where the ligand to metal ratio is at least 2:1. U.S. Patent application publication number 2005/0239763 (Motyka et al.) describes a metal amino acid chelate composition having an amino acid to metal molar ratio of from about 1:1 to 4:1, as does U.S. Patent application publication number 2005/0239750 (Motyka et al.). U.S. Patent application publication number 2003/0206969 discloses an iron/amino acid chelate having a metal:ligand ratio of 1:1-3.

Amino acids are generally considered to be efficiently absorbed in the normal human or animal intestine, but not all ingested amino acids are well-absorbed. For example, when pre-hydrolyzed bovine serum albumin (BSA) was perfused into the jejunum of human subjects, the amino acids alanine, phenylalanine, arginine, leucine, methionine, and tyrosine were absorbed at a rate of 50% or more (e.g., 86% for methionine). (Reicht, G. et al. (1997) Eur. J. Gastroenterol. Hepatol. 9: 81-85.) Other amino acids, such as cystine (3%) were absorbed at much lower rates. In individuals with certain diseases or conditions such as sepsis or peritonitis, those with muscle wasting disorders or cachexia related to AIDS or cancer, or older individuals in whom amino acid absorption decreases as part of the aging process, there is an even lower percentage of absorption of necessary amino acids. Certain amino acids are of particular interest because they promote protein synthesis, spare lean body mass during weight loss, decrease muscle wasting associated with aging, and promote wound healing. These are the branched-chain amino acids (BCAAs). BCAAs have received particular attention due to their effect on protein metabolism. After a meal containing protein has been ingested, studies have shown that half of the BCAAs from the meal will be absorbed and transported into the systemic circulation. (Matthews, D. (2005) J. Nutrition 135: 1580S-1584S).

Amino acid supplementation is generally accomplished by ingesting higher-protein foods, isolated proteins and/or amino acids, or hydrolyzed proteins. Aqueous formulations of the branched-chain amino acids, which would provide a formulation that could be more easily consumed by individuals who might particularly benefit from BCAA supplementation, are difficult to prepare because BCAAs have non-polar side groups and are hydrophobic.

What are needed are formulations containing branched-chain amino acids that make those amino acids more available to individuals in whom they may provide a benefit.

### SUMMARY OF THE INVENTION

The present invention relates to a mineral branched-chain amino acid chelate (BCAA/mineral chelate) and a method for preparing such a BCAA/mineral chelate. In one embodiment, the invention relates to a leucine chelate such as calcium leucinate.

In alternate embodiments, the mineral can comprise a mono- or divalent cation such as calcium, magnesium, potassium or zinc and the branched-chain amino acid can comprise isoleucine or valine.

Aspects of the invention comprise a method for making a BCAA/mineral chelate and a product (composition) made by the method, the method comprising admixing a branched-chain amino acid, a base, and a mineral. In some aspects, the method for admixing comprises the steps of sequentially admixing the mineral with the base to form a mineral/base solution, then admixing the mineral/base solution with the branched-chain amino acid to form the BCAA/mineral chelate. In some aspects, the mineral is chosen from the group consisting of calcium, magnesium, potassium, and zinc, the base is sodium hydroxide, and the branched-chain amino acid is chosen from among the group consisting of leucine, isoleucine, and valine.

One aspect of the invention relates to methods of incorporating branched-chain amino acids into food or nutritional supplement products. In one embodiment, a method for incorporating branched-chain amino acids into liquid or aqueous food or supplements is provided, the method comprising providing one or more branched-chain amino acids in the form of a BCAA/mineral chelate, particularly a BCAA/mineral chelate prepared by the method of the invention.

The invention also relates to methods of use of BCAA chelates to provide increased levels of BCAAs to a human or animal subject via food and/or nutritional supplementation. In one embodiment, nutritional supplementation may comprise a nutritional supplement product such as a powder, capsule, tablet, or nutritional drink, for example. In another aspect, nutritional supplementation may comprise supplementation of a food or feed product.

One aspect of a composition of the invention comprises a combination of one or more BCAA/mineral chelates and a whey protein product such as whey protein concentrate or whey protein isolate.

### DETAILED DESCRIPTION

The invention is a composition comprising a mineral/branched chain amino acid chelate formed by chelating a mineral such as calcium or magnesium using leucine or other branched chain amino acid as the chelating amino acid. In the method of the invention, a BCAA/mineral chelate is formed by admixing a mineral, a base, and a branched-chain amino acid. In some aspects, the method comprises first admixing the mineral with the base to form a mineral/base solution, then adding the branched-chain amino acid to the mineral/base solution. The composition may be formed by reacting a mineral source, such as, but not limited to, calcium or magnesium carbonate, with free leucine, or other branched chain amino acid (BCAA), to form, in one embodiment, calcium leucinate. In compositions of the present invention, the mineral can comprise, for example, calcium, magnesium, potassium or zinc and the branched-chain amino acid can comprise leucine, isoleucine or valine.

It should be understood that where “a” is used, such as “a” mineral, “a” base, and/or “a” branched-chain amino acid, aspects of the invention may comprise “at least one” mineral, base, and/or branched-chain amino acid and the term “a” is used only for convenience and simplicity and not intended to be limiting. A “base,” as used herein, is a chemical composition such as, for example, sodium hydroxide, magnesium hydroxide, or any suitable composition of basic pH.

The mineral moiety of a composition of the invention can be provided by, for example, calcium chloride, calcium carbonate, calcium citrate, calcium lactate, calcium gluconate, calcium phosphate, milk calcium, coral calcium, egg shell calcium, calcium hydroxide, calcium sulfate, magnesium chloride, magnesium carbonate, magnesium citrate, magnesium oxide, magnesium phosphate, magnesium hydroxide, magnesium lactate, magnesium sulfate, zinc sulfate, zinc citrate, zinc gluconate, zinc picolinate, zinc chloride, zinc oxide and/or other calcium, magnesium, zinc, or potassium salts. Other appropriate sources of mono- or divalent cations such as calcium, magnesium, zinc, or potassium, for example, are known to those of skill in the art and may be used in the present composition.

The present invention utilizes the mineral in a BCAA chelate to further increase ease of incorporation of the BCAA moiety of the chelate into a food or nutritional supplement product and to facilitate delivery of BCAAs to a human or animal subject. Especially where aqueous formulations are desired, or aqueous formulations are utilized in the preparation of more non-aqueous products, such as feeds, nutritional bars, and other more solid compositions, BCAA/mineral chelates of the invention provide an amino acid that is more easily incorporated into the final product.

In embodiments of the invention comprising calcium, the calcium may be provided in a variety of forms including, for example, a dairy mineral composition, calcium chloride or calcium hydroxide. For calcium leucinate compositions, for example, compositions of the present invention may comprise from about 75 to about 80 percent leucine and from about 10 to about 12 percent calcium. In one embodiment, a calcium leucinate composition comprises a calcium to leucine molar ratio of 1 to 2.

Depending upon the particular mineral/amino acid combination desired, compositions can comprise, by weight, from about 40% to about 90% amino acid and from about 5 to about 60 percent mineral. In some embodiments, compositions may comprise from about 10 to about 40 percent mineral in combination with from about 60 to about 90% amino acid. Compositions may also comprise from about 75 to about 80 percent amino acid and from about 20 to about 25% percent mineral.

Compositions of the invention may be incorporated into formulations including, but not limited to, a full range of beverages, bars, foods, animal feeds, powders, drinks, or provided as supplement tablets, caplets, capsules, or other formulations for human or animal use.

Compositions described by the invention may be used to provide increased availability of branched-chain amino acids, which are known to play important roles in a variety of metabolic processes. Branched-chain amino acids L-leucine (2-amino-4-methylvaleric acid; alpha-aminoisocaproic acid; (S)-2-amino-4-methylpentanoic acid, chemical formula C₆H₁₃NO₂), L-valine (2-aminoisovaleric acid; 2-amino-3-methylbutyric acid; alpha-aminoisovaleric acid; (S)-2-amino-3-methylbutanoic acid; chemical formula C₅H₁₁NO₂); and L-isoleucine (2-amino-3-methylvaleric acid; alpha-amino-beta-methylvaleric acid; (2S,3S)-2-amino-3-methyl pentanoic acid; chemical formula C₆H₁₃NO₂) play important roles, for example, in modulation of protein turnover and recovery from mental fatigue. For example, leucine has been shown to be a specific modulator of protein turnover (Bevington, A. et al. Eur. J. Clin. Invest. (2001) 31(6): 497-503) and branched-chain amino acids have been shown to decrease muscle proteolysis via inhibition of lysosomal proteolysis (Busquets, S. et al. J. Cell Physiol. (2000) 184(3): 380-4).

Branched-chain amino acids have been demonstrated to be significantly decreased after mental fatigue (J. Neural Transm. (2006) Dec. 11, 2006 e-publication) and administration of BCAAs was recently shown to decrease central fatigue after exercise (J. Nutr. (2006)136: 544S-547S).

Administration of compositions of the present invention provides a method for increasing the availability of BCAAs in food and nutritional supplements and related products and providing a therapeutic benefit for building and/or maintaining muscle tissue where loss of muscle tissue is associated with conditions that include, but are not limited to, stroke, spinal cord injury, osteoarthritis, rheumatoid arthritis, prolonged corticosteroid therapy, diabetic neuropathy, bums, amyotrophic lateral sclerosis (ALS), Guillain-Barre Syndrome, Muscular Dystrophy, sarcopenia associated with aging, and muscle tissue loss associated with weightlessness in astronauts.

Administration may be accomplished by oral or enteral means, and compositions may include tablets, caplets, gelatin capsules, powders, nutritional foods, nutritional drinks, enteral feeding formulations, and the like, comprising at least one BCAA/mineral chelate of the invention.

The invention provides nutritional therapies for individuals having the aforementioned conditions, as well as for athletes desiring to build and/or maintain muscle mass and improve muscle strength. The invention may also provide a nutritional means to decrease mental fatigue. In one embodiment, compositions of the invention may be provided as tablets, caplets, gelatin capsules, chewable tablets, a supplemented drink, nutrition bar, or other formulation to provide at least one BCAA/mineral chelate to an individual to decrease mental fatigue following, for example, strenuous exercise or physically-demanding work. BCAA/mineral chelates of the invention may also be administered in a method of the invention to increase BCAA absorption and decrease central fatigue after exercise.

Table 1 lists ingredients comprising an example of a composition comprising a powdered formulation to provide a nutritional drink as provided by the invention.

TABLE 1

| | Amount | Protein | Fat | Carb | Leucine | Total |
|---|---|---|---|---|---|---|
| Ingedient | (g) | (g) | (g) | (g) | (g) | BCAA |
| WPC 80 | 8.34 | 6.51 | 0.75 | 0.33 | 0.72 | 1.48 |
| WPI | 4.02 | 3.51 | 3.02 | 0.03 | 0.39 | 0.85 |
| Chelated leucine | 0.48 | | | | 0.40 | 0.63 |
| Crystalline Fructose | 17.00 | | | | | |
| Carageennan | 1.00 | | | | | |
| Brazilian Cocoa | 1.50 | | | | | |
| Powder | | | | | | |
| Color | 0.03 | | | | | |
| Salt | 0.06 | | | | | |
| Milk Flavor | 1.70 | | | | | |
| Chocolate Malt | 1.50 | | | | | |
| Flavor | | | | | | |
| Vanilla Flavor | 1.00 | | | | | |
| Total | 36.68 | 10.02 | 3.77 | 0.36 | 1.51 | 2.95 |
| percent of protein | | | | | 15.03 | 29.48 |
| 2 times per day | 73.37 | 20.04 | 7.53 | 0.72 | 3.01 | 5.91 |
| consumption | | | | | | |

In a formulation such as that shown in Table 1 above, it is understood that ingredient quantities and ratios may be varied significantly yet still provide a formulation that will be easily dispersed in water. WPC (whey protein concentrate), for example, may be present in such a formulation from about 5 grams to about 10 grams. Whey protein isolate (WPI) may be present from about 1 gram to about 7 grams. Chelated leucine may be present in such a composition from about 0.1 gram to about 2 grams, and crystalline fructose may be present from about 10 grams to about 20 grams.

Compositions of the invention may comprise at least about about 0.1 grams of leucine, isoleucine, valine, or combinations thereof, and preferably will be formulated to provide at least about 3 grams daily of leucine, isoleucine, valine, or combinations thereof. Compositions may be provided for occasional or for regular use, with regular use being preferred. Regular use may include daily, twice-daily, every other day, or other regimens, for example. For improved effect, administration of at least 3 grams of leucine per day, for example, may be preferred. Compositions of the invention, providing, for example, BCAA/mineral chelates for use in aqueous formulations, powders dispersed in aqueous formulations, or similar compositions, increase availability of BCAAs to human or animal subjects to whom they are provided or administered. Also disclosed is a method for providing branched-chain amino acids in a liquid beverage, the method comprising forming a branched-chain amino acid chelate and adding the chelate to the beverage.

The invention may be further described by means of the following non-limiting examples.

### EXAMPLES

The inventors attempted to incorporate free leucine and micro-encapsulated free leucine into beverages and found both products difficult to use and difficult to disperse. The microencapsulated leucine produced a turbid, cloudy solution, indicative of non-dissolving particulates. Free leucine showed a separation phenomenon in aqueous solution. When a BCAA/mineral chelate of the invention was utilized, however, the chelated leucine product was much more easily dispersed and did not separate or increase turbidity of aqueous systems.

### Sample Calcium Leucinate Formulation

To form calcium leucinate, 26.2 grams of L-leucine (Seltzer Ingredients, Carlsbad, Calif.) were dispersed in 75 ml deionized water. While stirring, 8 grams of sodium hydroxide were added. The solution was heated to approximately 42 degrees C. to dissolve the leucine. In a separate container, 11.1 grams of calcium chloride (Chris Hansen) were dissolved in 25 ml deionized water. While stirring the leucine solution, the calcium chloride solution was added. A voluminous white precipitate was formed. The precipitated calcium-leucine chelate was separated, washed and dried.

What is claimed is:

1. A composition comprising a branched-chain amino acid/mineral chelate, wherein the mineral to amino acid ratio is 1:2.

2. The composition of claim 1 wherein the mineral is chosen from the group consisting of calcium, magnesium, potassium, and zinc.

3. The composition of claim 1 wherein the branched-chain amino acid is chosen from the group consisting of leucine, isoleucine, and valine.

4. The composition of claim 1 wherein the amino acid comprises from io about 75 to about 80 percent by weight and the mineral comprises from about 20 to about 25% percent.

5. The composition of claim 1 wherein the amino acid comprises from about 40 to about 90 percent by weight and the mineral comprises from about 5 to about 60 percent.

6. The composition of claim 1 wherein the amino acid comprises from about 60 to about 90 percent by weight and the mineral comprises from about 10 to about 40 percent.

7. The composition of claim 1 further comprising a whey protein isolate or whey protein concentrate.

8. A composition comprising a branched-chain amino acid/mineral chelate produced by a method comprising admixing a mineral, a base, and a branched-chain amino acid.

9. The composition of claim 8 wherein the step of admixing is performed sequentially, by a first step of admixing the mineral with the base to form a mineral/base solution and a second step of admixing the mineral/base solution with the branched-chain amino acid to form the branched-chain amino acid/mineral chelate.

10. The composition of claim 8 wherein the base is sodium hydroxide.

11. The composition of claim 8 wherein the branched-chain amino acid is chosen from the group consisting of leucine, isoleucine, and valine.

12. The composition of claim 8 wherein the mineral is chosen from the group consisting of calcium, magnesium, potassium, and zinc.

13. A method for forming a branched-chain amino acid/mineral chelate, the method comprising admixing a mineral, a base, and a branched-chain amino acid.

14. The method of claim 13 wherein the step of admixing is performed sequentially, by a first step of admixing the mineral with the base to form a mineral/base solution and a second step of admixing the mineral/base solution with the branched-chain amino acid to form the branched-chain amino acid/mineral chelate.

15. The method of claim 13 wherein the base is sodium hydroxide.

16. The method of claim 13 wherein the branched-chain amino acid is chosen from the group consisting of leucine, isoleucine, and valine.

17. The method of claim 13 wherein the mineral is chosen from the group consisting of calcium, magnesium, potassium, and zinc.
